(19) United States
(12) Patent Application Publication
(10) Pub. No.: US 2018/0229202 A1
(43) Pub. Date: Aug. 16, 2018

(54) DEVICE FOR INJECTING FLUIDS INTO THE FREE AREA OF A ROTATING FLUIDISED BED

(71) Applicant: Axel De Broqueville, Grez-Doicceau (BE)

(72) Inventor: Axel De Broqueville, Grez-Doicceau (BE)

(21) Appl. No.: 15/753,194

(22) PCT Filed: Aug. 2, 2016
(86) PCT No.: PCT/BE2016/000036

§ 371 Date: Feb. 16, 2018

(30) Foreign Application Priority Data
Aug. 24, 2015 (BE) BE2015/0206

(51) Int. Cl. B01J 8/38; B01J 8/18; B01J 8/00; C07C 5/48; B04C 9/00

(52) U.S. Cl. CPC B01J 8/386; B01J 8/1827; B01J 8/0055; B04C 2009/008; B04C 9/00; B01J 2208/00761; B01J 2208/00893; C07C 5/48

(57) ABSTRACT

Device for injecting fluids into the free area of a rotating fluidized bed revolving in a fixed cyclone chamber, and method using this device, comprising a device for tangentially injecting secondary fluids, enabling rotating rings of fluids to be formed in said free area along the side walls of said cyclone chamber, in order to separate from said side walls fluid flows exiting along said side walls and accelerate their rotation velocity, and thus to improve the retention of the solid particles entrained by said exiting fluid flows

### TECHNICAL FIELD OF THE INVENTION

The present invention relates to devices for improving the separation of the fluids and solid particles forming a rotating fluidized bed revolving inside a fixed cyclone chamber, and to methods for processing fluids and solids using these devices.

### TECHNICAL BACKGROUND

The use of centrifugal force for separating solid particles from fluids in cyclonic separators or cyclones is well known. With the aim of reducing the entrainment of solid particles along the fixed walls toward the central fluid outlet, the patent DE 10 2008 056 952 A1 describes a device for injecting air around the central fluid discharge tube to form a rotating ring that separates the solid particles from the central tube, and the European patent application EP 1,958,699 A1 describes a device for injecting fluid along the circular wall to improve the segregation of the particles. These devices do not eliminate the entrainment of solid particles coming from the side opposite the central tube through an area around the axis of symmetry, where the centrifugal force is very weak. To overcome this, the cyclonic separator is usually vertical, and the ratio between the length or height of the cyclonic separator and its diameter is large, being preferably greater than 3. The secondary air injection device as described in the patent DE 10 2008 056 952 A1 may also generate turbulent backflow, entraining the solid particles toward the central tube.

The forming of rotating fluidized beds in a fixed cyclone chamber, or “vortex” chamber, is known. The centrifugal force of the free vortex revolving in the free area, surrounded by the rotating fluidized bed, increases more rapidly than the inverse of the square of the radius, enabling the solid particles to be separated from the lighter fluids. However, the production of dense, stable rotating fluidized beds with fine particles or powders requires narrow cyclone chambers, the width of which is preferably smaller than its radius, in order to avoid the formation of channels through which the fluids may escape while having very little contact with the solid particles. This narrowness increases the effect of the fixed surfaces in contact with said free vortex. The friction in the central tube and along the side walls of the fixed cyclone chamber decreases the rotation speed of the fluids, promoting losses of solid particles which travel along the side walls where the rotation velocity is lower and the radial velocity is higher. Furthermore, the production of dense, stable rotating fluidized beds with powders requires very high fluid flow rates and a very high centrifugal force, preferably hundreds of times greater than the force of gravity, entailing high energy consumption and causing attrition of the powders.

Mechanical rotating devices such as a rotating disk described in the patent BE 1020683 or a rotating central flue, described in the U.S. Pat. No. 8,257,657 in the name of the same inventor, may be used to improve the separation of the fluids and particles in a rotating fluidized bed. These solutions have the drawbacks associated with mechanical devices revolving at high speed inside cyclone chambers.

The present invention enables the rotation velocity of the fluids to be increased in the free area of the rotating fluidized bed, mainly along the side walls, thereby avoiding the entrainment of the solid particles along said side walls, without requiring high rotation velocities of the rotating fluidized bed, while avoiding the mechanical constraints associated with the use of rapidly revolving objects in cyclone chambers. It may also be used to provide rotating rings of fluids that are sufficiently uniform to generate little troublesome turbulence. It may also contribute to the improvement of methods for processing fluids and/or solids using cyclone chambers.

### BRIEF DESCRIPTION OF THE INVENTION

The present invention is a device for injecting secondary fluids into the free area of a rotating fluidized bed in a fixed cyclone chamber, comprising a peripheral wall (4) and two side walls (3) and (5); a device for supplying fluid (57) through openings (72) distributed along said peripheral wall, in a mainly tangential direction (73); a device for supplying and discharging solid particles, (59) and (63), which are usually entrained by a fluid, through said side walls or said peripheral wall; and at least one central tube (7) for discharging the fluids revolving in the cyclone chamber through a said wall, characterized in that it comprises a device for supplying secondary fluids (17) and (17.1) through central supply chambers (15) and (15.1) located along each of said side walls, having injection openings (18) and (18.1) distributed around the axis of cylindrical symmetry (1), enabling said secondary fluids to be injected in a mainly tangential direction (19) and (19.1), in order to form two rotating rings (44) and (44.1) of secondary fluids revolving around the axis of cylindrical symmetry (1), along said side walls, within said free area, and in that said devices for supplying said secondary fluids enable said secondary fluids to be supplied at a sufficient pressure to make said rotating rings of secondary fluids revolve at a velocity which is greater than, and preferably at least one and a half times greater than, the highest rotation velocity of said fluids revolving in the free area when the latter is in operation without the device for injecting secondary fluid according to the invention.

In order to form a stable, dense rotating fluidized bed, the mean distance between said two side walls is usually less than the mean diameter, and preferably less than the mean radius of said peripheral wall, and the distance between the openings (72) for supplying fluid (59) through said peripheral wall is usually smaller than, and preferably smaller than half of, its mean radius.

According to a particular embodiment of the invention, said central chamber (15.1) for supplying a secondary fluid (17.1), fixed to said side wall (5), opposite said central tube (7), surrounds another central tube (7.1) for discharging the fluids.

According to another particular embodiment of the invention, said cyclone chamber, comprising at least one said central tube for discharging the fluids, each such tube passing through said side walls, is divided into two transverse sections, A and B, by a separating wall (53), having at least one passage (39) along said peripheral wall and having a central part comprising at least one central supply chamber (15.3) and (15.4), connected to one or more tubes (13.2) and (13.3), for supplying one or more secondary fluids (17.2) and (17.3) injected through injection openings (18.2) and (18.3) in a mainly tangential direction (19.2) and (19.3) around the axis of cylindrical symmetry (1), along both sides of said separating wall (53), in said free areas of said transverse sections.

According to a preferred form of this particular embodiment of the invention, said passages (39) are distributed alternately so as to coincide with the upstream end of a said opening (72) of a transverse section and the downstream end of a said opening (72) of the other transverse section. Thus the static pressure difference between the upstream and downstream ends increases the transfer of the solid particles from one transverse section to the other.

The size and number of said passages (39) allowing the solid particles entrained by fluids to pass from one said transverse section to the other, and the sizes of said two transverse sections A and B, are to be determined according to the requirements of the method that uses said device.

The high-speed injection of said secondary fluids via said injection openings generates periodic variations of static and dynamic pressure, and therefore of the turbulence, which is transmitted to the fluids revolving in the cyclone chamber. This turbulence is usually undesirable, as it may facilitate the entrainment of the solid particles toward the central tube. For this reason, according to a preferred embodiment of the invention, the desirable number of said injection openings is at least 8, and preferably less than 12, for at least one and preferably for each of said central supply chambers. According to this preferred embodiment of the invention, at least one and preferably each of said rotating rings of secondary fluids is formed behind guide walls, from (38) to (38.3), which may be the side walls (3) or (5), or may be a rotating disk (50), in order to improve their uniformity before coming into contact with the fluids rotating in the cyclone chamber.

When the device according to the invention is in operation, said rotating rings of secondary fluids, revolving within the cyclone chamber more rapidly than the neighboring fluids, are pushed by centrifugal force along said side walls and said separating wall toward said peripheral wall, thus forming a backflow of secondary fluids, from (35) to (35.3), generating vortices, hereafter called secondary toroidal vortices, from (36) to (36.3), revolving around the axis of symmetry (1) and also around circular areas (27) within the axial sections of said secondary toroidal vortices.

Said backflows of secondary fluids revolving around the axis of symmetry (1) more rapidly than the exiting fluids from (30) to (30.3), which flow along said side and separating walls while entraining solid particles, separate said exiting fluid flows from said side and separating walls and increase their rotation velocity. If the rotation velocity of said backflows of secondary fluids is high enough, the higher centrifugal force retains toward said peripheral wall a part, called the retained part, from (33) to (33.3), of said exiting fluid flows with most of the solid particles entrained by said exiting fluid flows, thus forming or strengthening other toroidal vortices (32) which revolve around the axis of symmetry of the cyclone chamber and also around circular areas (27) within the axial sections of said toroidal vortices. The other part, called the discharged part, from (34) to (34.3), of said exiting fluid flows, having released most of the solid particles, bypasses said secondary toroidal vortices. One part, called the recycled part (9) and (9.1), is recycled with the secondary fluids, and the other part, called the centrally exiting fluids (6), is discharged centrally via said central tube or tubes.

The retention force of said backflows of secondary fluids, which is proportional to the square of the rotation velocities of said rotating rings of secondary fluids, is also proportional to the flow rate of said secondary fluids, which must be high enough to provide significant retention, since the transfer of kinetic moment of rotation becomes inefficient when the differences in velocity are very large. It is therefore desirable to have a flow rate of said secondary fluids in each of said central supply chambers that is sufficiently high, being at least 5% and preferably at least 8% of the flow rate of the fluids passing through the cyclone chamber.

According to a particular embodiment of the invention, said side wall (5), opposite said central tube (7), is equipped with a lateral rotating disk (50) for guiding said rotating ring of secondary fluid (17.1) by revolving around the axis of symmetry of the cyclone chamber, near said side wall (5), the disk being driven by a rotation device that can make it revolve at a higher velocity than the rotation velocity of the fluids revolving in the free area, in order to increase the retention force of the backflow of secondary fluid (35.1) that generates the secondary toroidal vortex (36.1). The dimensions of said rotating disk and its rotation velocity may be determined according to the requirements of the method for which the cyclone chamber is used.

The rotation velocity of the centrally exiting fluids (6) is reduced by friction in the central tube or tubes, which decreases the centrifugal force there and generates a virtually non-rotating central backflow (41) along the axis of cylindrical symmetry (1). According to a preferred embodiment of the invention, said central tube or tubes are short, preferably having a length smaller than their diameter, and terminate in a cylindrically symmetric conduit (21), which enables the centrally exiting fluids to be discharged tangentially, in order to reduce the deceleration of the rotation velocity of said centrally exiting fluids. Said cylindrically symmetric conduit (21) is delimited on one side by the flared surface (20) of the end of said central tube, and on the other side by a lateral disk (22) whose central part has a curved surface (26) which progressively deflects the centrally exiting fluids, called the deflected fluids (28), without impeding their rotation around the axis of symmetry (1). The profile of said curved surface (26) of said lateral disk (22) preferably takes the form of a cone with a curved profile, the tip (40) of which may be rounded, while its base is preferably flared.

According to a particular embodiment of the invention, said tip (40) of the curved surface (26) of said lateral disk (22) penetrates into said central tube, as far as the inlet and preferably beyond the inlet of said central tube, in order to push said central backflow (41) back into the cyclone chamber.

Said cylindrically symmetric conduit (21) is closed by a circular wall (23), which is preferably narrow and has an internal width (49) which is less than the radius of the inlet of said central tube and is preferably less than half of said radius. Said circular wall (23) is equipped with at least two tangential output openings (24) for tangentially discharging, in the direction of rotation of the fluids, said deflected fluids (28) that revolve around the axis of cylindrical symmetry (1) in the cylindrically symmetric conduit (21).

A large number of tangential output openings (24) makes it possible to reduce the periodic turbulence which is undesirable in said central tube and is transmitted to the inside of the cyclone chamber. It is therefore desirable to have at least 4, and preferably at least six of these openings. The sum of the cross sections of said tangential outlet openings (24) is smaller than, and preferably at least twice as small as, the cross section of the inlet of said central tube, and preferably small enough for the mean velocity of the discharge of the fluids through said tangential outlet openings (24) to be greater than the total mean velocity of the centrally exiting fluids (6) at the inlet of said central tube, when the device is in operation. Thus the rotation velocity around the axis of symmetry (1) of the centrally exiting fluids is not reduced by said central tube.

The high kinetic energy of the discharged fluids may be partially recovered, for example if they directly supply a turbine. According to a preferred form of this particular embodiment of the invention, said tangential outlet openings (24) are prolonged by flared conduits (42) whose outlet cross section is at least twice, and preferably at least three times, greater than the cross section of said tangential outlet openings (24), to decelerate the discharged fluids in order to recover some of their kinetic energy. Said flared conduits (42) may be connected to one or more manifolds (89).

According to another particular embodiment of the invention, the device for supplying secondary fluids comprises two contiguous central supply chambers (15.1) and (14), allowing the injection of different secondary fluids, one being gaseous and the other being liquid, in order to spray droplets of the secondary liquid into said rotating ring (44.1) of secondary fluids. Said droplets of said secondary liquid may be used to react with said exiting fluid flows and/or said solid particles entrained by said exiting fluid flows.

According to another particular embodiment of the invention, said central tube (7) for discharging the fluids contains one or more concentric tubes (80) which penetrate to a greater or lesser extent into the cyclone chamber. According to this particular embodiment of the invention, said concentric tube or tubes may serve to discharge or supply fluids centrally, or may serve to spray a liquid (86).

According to a particular embodiment of the invention, the device for discharging the solid particles comprises at least one decantation tube (91) passing through said peripheral wall and allowing the solid particles (63) to accumulate under the effect of their inertia and gravitational force when the device is operating. Said decantation tube may be equipped with an inlet and outlet valve which opens alternately, or with a rotary valve, for discharging the accumulated solid particles at the desired rate, without entraining the fluids revolving in said cyclone chamber.

The present invention also relates to the use of the device according to the invention by methods for processing solid particles reacting in contact with the fluids passing through the cyclone chamber, and by methods for processing fluids passing through the cyclone chamber in contact with the solid particles, characterized in that a secondary fluid injected by a device according to the invention is used to retain the solid particles entrained by said fluid flows exiting along said side walls of the cyclone chamber.

The methods for processing solid particles reacting in contact with the fluids passing through the cyclone chamber comprise, for guidance and in a non-limiting way, the combustion, gasification, pyrolysis, roasting, drying, impregnation, surface treatment, encapsulation, polymerization, oxidation and reduction of said solid particles. The methods for processing fluids passing through the cyclone chamber in contact with solid particles comprise, for guidance and in a non-limiting way, the cracking, dehydrogenation, alcohol to olefin conversion, disproportionation, oxidation and reduction of said fluids. The solid particles may be, or may contain, catalysts.

The device for injecting secondary fluids and the preferred forms of central discharge of the fluids in a cyclone chamber, according to the invention, enable the rotation velocity of the fluids in the free area of the rotating fluidized bed to be increased, without a significant increase in the rotation velocity of said rotating fluidized bed. It is therefore particularly suitable for methods in which the rotation velocity of the solid particles along the circular wall is limited by mechanical or physical constraints, such as abrasion, excessive weight of the rotating fluidized bed, the fragility of the solid particles, or other undesirable effects.

The improvement of the retention of the solid particles entrained by the fluid flows exiting along the side walls makes it possible to use solid particles that are smaller, and therefore more reactive. The greater reactivity of the solid particles makes it possible to increase the flow rate and thus reduce the dwell time of the revolving fluids in the cyclone chamber. The reduction of the dwell time of the fluids makes it possible to increase the reaction temperature and/or to reduce undesirable secondary reactions, because of the rapid cooling of said exiting fluid flows by said secondary fluids. For example, the catalytic cracking of hydrocarbons at high temperatures to produce olefins generates numerous undesirable secondary reactions. The dwell time of said hydrocarbons in a cyclone chamber may be less than 0.1 second, compared with the usual time of more than a second in existing fluidized catalytic crackers.

The present invention also relates to methods for processing solid particles and fluids revolving in a cyclone chamber comprising a device for injecting secondary fluid according to the invention, characterized in that at least one said secondary fluid reacts with at least one of the exiting fluid flows, or with the solid particles entrained by said exiting fluid flow along a said side wall or separating wall.

The term “react” comprises, for guidance and in a non-limiting way, chemical reactions such as oxidation and reduction reactions, and physical reactions such as cooling, impregnation, surface treatment and encapsulation.

For example, the combustion of carbonaceous matter in a rotating fluidized bed must sometimes be incomplete because of the limitation of the combustion temperature to avoid or limit slag formation. The oxygen contained in a secondary fluid may complete the combustion, which generates very high temperatures without significantly increasing the temperature of the solid particles revolving along the peripheral wall of the cyclone chamber.

A particular embodiment of the invention is a method for the combustion of fluid or solid carbonaceous matter revolving in a cyclone chamber, characterized in that the fluids injected tangentially (73) through the circular wall (4) do not contain enough oxygen to provide complete combustion of said carbonaceous matter, and in that at least one said secondary fluid supplied by a device according to the invention contains the necessary oxygen to complete the oxidation of said fluid flows exiting along a side wall.

The present invention also relates to methods for processing solid particles and fluids revolving in a cyclone chamber, characterized in that it comprises at least two said contiguous central supply chambers, one injecting a secondary liquid and the other injecting a secondary gas, via one of the devices according to the invention, said secondary liquid reacting with at least one of said fluid flows or with the solid particles entrained by said flow of fluids exiting along a said side wall or separating wall.

For example, in order to obtain very rapid cooling, said secondary liquid may be a cooling liquid, entrained and dispersed by said secondary gas in said secondary toroidal vortex. The droplets of liquid may mix with the flow of hot gases passing along the side walls toward the central tube, and cool the hot gas, evaporating even before said hot gases have left the cyclone chamber.

Said secondary liquid may also be used to react with the solid particles entrained by said fluid flows (30) exiting along the side wall, for example by impregnating them or surface treating them, thereby making them heavier and improving the retention of said solid particles back toward said peripheral wall.

The present invention also relates to methods for processing fluids and solid particles comprising the alternating processing of the solid particles revolving in a cyclone chamber divided into two transverse sections, A and B, separated by a separating wall (53) with different reactions in each section, for example an endothermic reaction in one section and an exothermic reaction in the other section, characterized in that at least one said secondary fluid injected by a device according to the invention is used to react with at least one of the fluid flows or with the solid particles entrained by said fluid flow exiting along a said side wall or separating wall.

For example, the carbon deposited on the catalysts in the catalytic cracking of hydrocarbons in one section may be oxidized in the other section, or, for example, the catalysts containing metal oxides for the dehydrogenation by oxidation and reduction of ethyl benzene in styrene or for the oxidation of carbonaceous matter may be reduced in one section and reoxidized in the other section. The secondary fluids may, for example, oxidize the exiting fluids, or cool them rapidly to avoid secondary reactions.

### BRIEF DESCRIPTION OF THE DRAWINGS

FIG. 1 shows an axial section through an example of a device for injecting secondary fluids along the side walls in the free area of a rotating fluidized bed in a cyclone chamber and a preferred mode of discharge of the fluids revolving in said cyclone chamber.

FIG. 2 shows an axial section through another example of a device for injecting secondary fluids, in which said injection openings (18) and (18.1) are located on the outside of the cyclone chamber, along the side walls (3) and (5), and with a central fluid discharge tube on each lateral side of said cyclone chamber.

FIG. 3.a shows a transverse section through the device for injecting secondary fluids, along the cutting line AA′ of FIG. 2.

FIG. 3.b shows a transverse section through the device for central discharge of the fluids revolving in the cyclone chamber, along the cutting line BB′ of FIG. 2.

FIG. 4 shows an axial section through another example of the devices for injecting secondary fluids, with a lateral rotating disk (50) near said lateral side (5) of a cyclone chamber.

FIG. 5 shows an axial section through a cyclone chamber divided into two transverse sections A and B by a separating wall (53) having devices for injecting secondary fluids along its two sides and an injection device with two contiguous central supply chambers (15.1) and (14) for injecting different secondary fluids.

### DEFINITIONS

Cyclone chamber, or turbulence chamber, also called “vortex” chamber, signifies a chamber of circular, generally cylindrical shape, into which one or more fluids are supplied tangentially in order to make solid particles, supplied with said fluids or separately, revolve rapidly inside said chamber. Said fluids are discharged centrally after being separated by centrifugal force from said solid particles, which are discharged separately. A particular example of a cyclone chamber is a simple cyclonic separator, also called a cyclone. Cyclonic separators are usually very elongated, with a length preferably at least three times their diameter; their axis of cylindrical symmetry is usually vertical, and their lower part usually takes the form of a very elongated cone, at the bottom of which the solid particles are collected by gravitation.

A cyclone chamber with rotating fluidized bed is a cyclone chamber whose peripheral wall is provided with fluid supply openings, allowing the tangential injection of a fluid that passes through a fluidized bed of solid particles suspended in said fluid and revolving along said peripheral wall. The distance between the two side walls is usually small, preferably being less than the diameter, and even less than the radius, of the peripheral wall. The distance between said fluid supply openings is usually less than the radius, and preferably less than half the radius, of the peripheral wall. The recovery of the solid particles preferably takes place along, or near, the peripheral wall.

Fluid signifies a liquid or a gas, or a mixture thereof. If the cyclone chamber is equipped with a device for atomizing droplets of a liquid, the other fluids are usually gases.

Fluid flow is the turbulent flow of fluids, that may entrain solid particles, in approximate mean directions, represented purely for guidance by arrowed lines.

Toroidal vortex or vortex of the toroidal type refers to complex vortices that revolve both around the axis of cylindrical symmetry (1) and around themselves, that is to say around a central circular area (27) of approximately toroidal shape. The sections of these complex vortices are shown schematically by closed curves, which are a highly simplified representation, purely for guidance, of the complex turbulent circulation of the fluids revolving inside cyclone chambers.

Free area: if a rotating fluidized bed is formed in a cyclone chamber, the solid particles suspended in the fluid that passes through said fluidized bed are concentrated by centrifugal force in a relatively thin area along the peripheral wall, while the central area, called the free area, contains few solid particles, because the Coriolis force increases the centrifugal force here and retains the solid particles toward the peripheral wall.

The free surface of the rotating fluidized bed, also called the “separation surface”, is the surface separating the rotating fluidized bed from the free area. In reality, this surface is ill-defined and turbulent. Its section is represented hereafter by a wavy line (98).

A mainly tangential direction at any point is a direction of which the component tangential to the circumference centered on the axis of cylindrical symmetry (1) and passing through this point is larger than all the other components added together.

### DETAILED DESCRIPTION

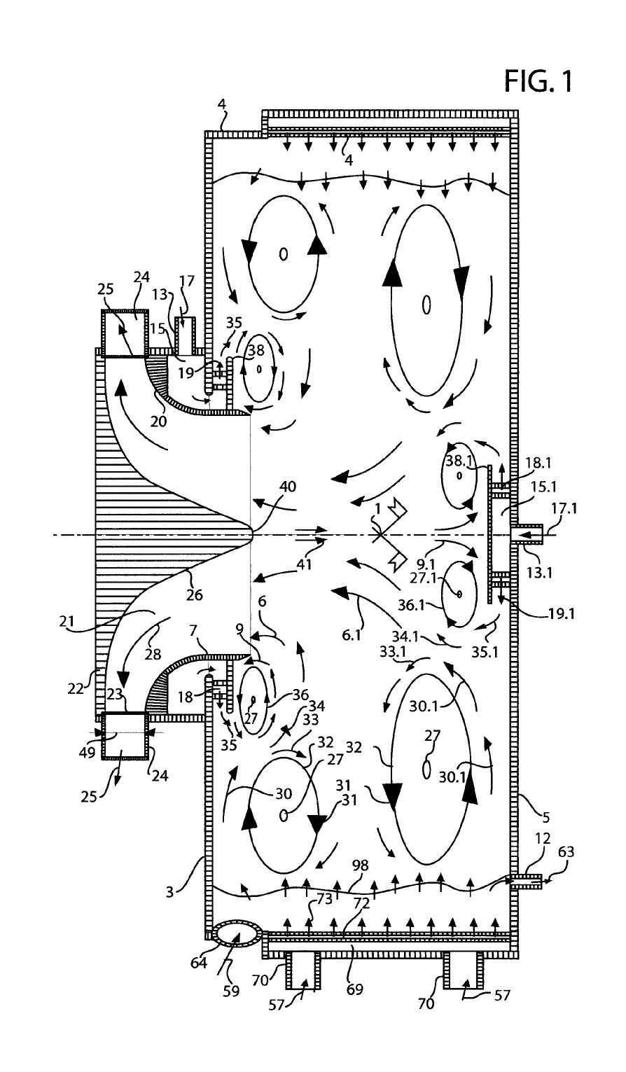

FIG. 1 shows an axial section through a cyclone chamber surrounded by a peripheral wall (4) and two side walls (3) and (5), comprising
a device for supplying solid particles (59), that may be entrained by a fluid, via a tube (64) passing through said peripheral wall and a device for discharging solid particles (63) via a tube (12) passing through the side wall (5) at a certain distance from said peripheral wall;
a device for supplying fluid (57) into a supply chamber (69) surrounding said peripheral wall, via tubes (70), enabling said fluids to be injected through a large number of openings (72), represented in the form of longitudinal slots, in a mainly tangential direction (73), that is to say a direction mainly perpendicular to the plane of the figure, in said peripheral wall, enabling a rotating fluidized bed to be formed;
a device for discharging the fluids (25) via a central tube (7) passing through the side wall (3); characterized in that it comprises:
a device for supplying secondary fluids (17) and (17.1) via the tubes (13) and (13.1) through central supply chambers (15) and (15.1), which are arranged along each side wall (3) and (5), one around said central tube (7) and the other around the axis of cylindrical symmetry (1), and are provided with openings, represented by the slots (18) and (18.1) distributed around said axis of cylindrical symmetry and arranged inside the cyclone chamber in this example, for injecting said secondary fluids (17) and (17.1) in mainly tangential directions (19) and (19.1) along said side walls (3) and (5), inside said free area, in order to form rotating rings of fluid behind guide walls (38) and (38.1).

When the cyclone chamber is in operation without the device for injecting secondary fluids according to the invention, the centrifugal force of the fluids revolving around the axis of cylindrical symmetry (1) concentrates most of the solid particles (63) in the rotating fluidized bed, the separation surface between said bed and said free area being represented by the wavy line (98) along said peripheral wall. Said solid particles (63) are discharged via the tube (12), the distance of which from said peripheral wall determines the mean distance of said free surface and said peripheral wall, and therefore the mean thickness of the rotating fluidized bed.

The friction along said side walls reduces the rotation velocity of the fluids around the axis of cylindrical symmetry (1), thereby reducing the centrifugal force and promoting the entrainment of solid particles by the fluid flows (30) and (30.1) exiting along said side walls. The centrifugal force generated by the tangential injection (73) of the fluids is higher at a certain distance from the side walls, and may generate a backflow of fluids (31) toward said peripheral wall, thus forming toroidal vortices (32) and (32.1) of fluids revolving both around the axis of cylindrical symmetry (1) and around a circular area (27) in the center of the axial sections of said toroidal vortices. This backflow of fluid reduces the rotation velocity of the fluids and increases the entrainment of the solid particles toward the central outlet by the fluid flows (30) and (30.1) exiting along the side walls.

When the device according to the invention supplies said secondary fluids at a sufficient pressure, the centrifugal force generated by the high rotation velocity of said rotating rings of secondary fluids generates a backflow of secondary fluids (35) and (35.1) along said side walls, separating said exiting fluid flows (30) and (30.1) from said side walls and accelerating their rotation velocity, a part, called the retained part (33) and (33.1), of these fluid flows containing most of the solid particles entrained by said exiting fluid flows, is retained toward the middle of said peripheral wall. The other part, called the discharged part (34) and (34.1), which has practically ceased to contain solid particles, is discharged toward the central tube (7) while mixing with the secondary fluid. A small part, called the recycled part (9) and (9.1), attracted by the low pressure generated by the centrifugal force of said rotating rings of secondary fluids, is recycled and mixes with the backflow of secondary fluids (35) and (35.1), thus forming a vortex called the secondary toroidal vortex (36) and (36.1), which revolves both around the axis of cylindrical symmetry (1) and around a circular area (27) inside the axial sections of said secondary toroidal vortex. These sections and these vortices vary in shape. They are shown schematically, purely for guidance.

The rotation velocity of said retained parts of said fluid flows exiting along the side walls, charged with solid particles, is increased by said secondary toroidal vortices, thereby improving the separation of the solid particles from the fluids passing through the rotating fluidized bed.

It must be possible for the injection pressure of the devices for supplying secondary fluids to be high enough to enable the injection velocity of the secondary fluids to be higher, and preferably at least one and a half times as high, as the highest rotation velocity of the fluids flowing centrally (6) from said cyclone chamber, when it is in operation without the device according to the invention.

The kinetic moment of rotation transferred by said secondary toroidal vortices to the exiting fluid flows depends on the injection velocity, and therefore on the injection pressure, as well as on the flow rate of said secondary fluids, and therefore on the total cross section of the injection openings (18). These quantities may be chosen according to the requirements and constraints of the method using this device. If the function of the secondary fluids is solely to improve the quality of separation of the fluids and fine solid particles, a low flow rate, with high injection velocities, and therefore a high injection pressure through injection openings with small cross sections, is preferable. However, the transfer of the kinetic moment of rotation becomes inefficient when the differences in velocity are very large. It is therefore desirable to have a flow rate of said secondary fluids that is sufficiently high, preferably in each of said central supply chambers, being at least 5% and preferably at least 8% of the flow rate of the fluids passing through the cyclone chamber.

When the fluids are discharged via an ordinary central tube (7), the friction along and the tube and at its end reduces the rotation velocity of the fluids and consequently the centrifugal force inside said central tube, thereby reducing the centrifugal pressure drop along the axis of symmetry and generating a pressure gradient toward said cyclone chamber, and consequently a virtually non-rotating central backflow (41), which reduces the rotation velocity of the fluids in said cyclone chamber.

The secondary toroidal vortices (36) and (36.1) increase the rotation velocity around the axis of symmetry (1) of the centrally exiting fluids (6) in the central tube (7), and therefore also increase the losses due to friction inside the central tube (7) and the undesirable central backflow (41) toward said cyclone chamber. In order to reduce these losses of rotation velocity, the central tube (7) for discharging the centrally exiting fluids (6) is short, its length preferably being less than the diameter of its inlet. Its outer end is flared and terminates in a cylindrically symmetric conduit (21), delimited on one side by the flared surface (20) of said central tube and on the other side by the curved central surface (26) of a lateral disk (22) closing the axial outlet of said central tube.

Said cylindrically symmetric conduit (21), for deflecting the centrally exiting fluids (6), called deflected fluids (28), without preventing their rotation around the axis of symmetry (1), by converting their axial velocity (29) to radial velocity, terminates in a circular wall (23), comprising at least two and preferably at least six tangential outlet openings (24) for discharging the fluids (25) in a mainly tangential direction through said circular wall (23). Said curved surface (26) has a conical profile, with a rounded tip (40) and flaring at the base, chosen so that the cross sections of said cylindrically symmetric conduit (21) are smaller than the inlet of said central tube (7), to progressively deflect the exiting fluids without slowing them.

The inside width (49) of said circular wall (23) is less than the radius, and preferably less than half the radius, of the inlet of said central tube (7), so that the radial outlet velocity of the discharged fluids (25) through said circular wall (23) is relatively high. The sum of the cross sections of said tangential outlet openings (24) is smaller than half the inlet cross section of the central tube (7), and preferably small enough for the outlet velocity of said discharged fluids (25) to be higher than the total mean velocity of the centrally exiting fluids (6) at the inlet of the central tube (7).

Said tip (40) of said curved surface penetrates into said central tube (7), preferably beyond the inlet of said central tube (7), in order to push the initiation of the central backflow (41) back into the cyclone chamber. Said central backflow (41), virtually non-rotating, is drawn in and recycled (9.1) by the low pressure generated by said secondary toroidal vortex (36.1) which transfers some of its kinetic moment of rotation to it before being discharged via the central tube (7). This transfer of kinetic moment of rotation reduces the negative effect of said central backflow on the rotation velocity of the fluids inside the cyclone chamber.

The central discharge of the fluids according to the preferred embodiment of the invention, described in this example, thus makes it possible to reduce and even reverse the negative effect of the central fluid discharge tube on the rotation velocity of the fluids inside said cyclone.

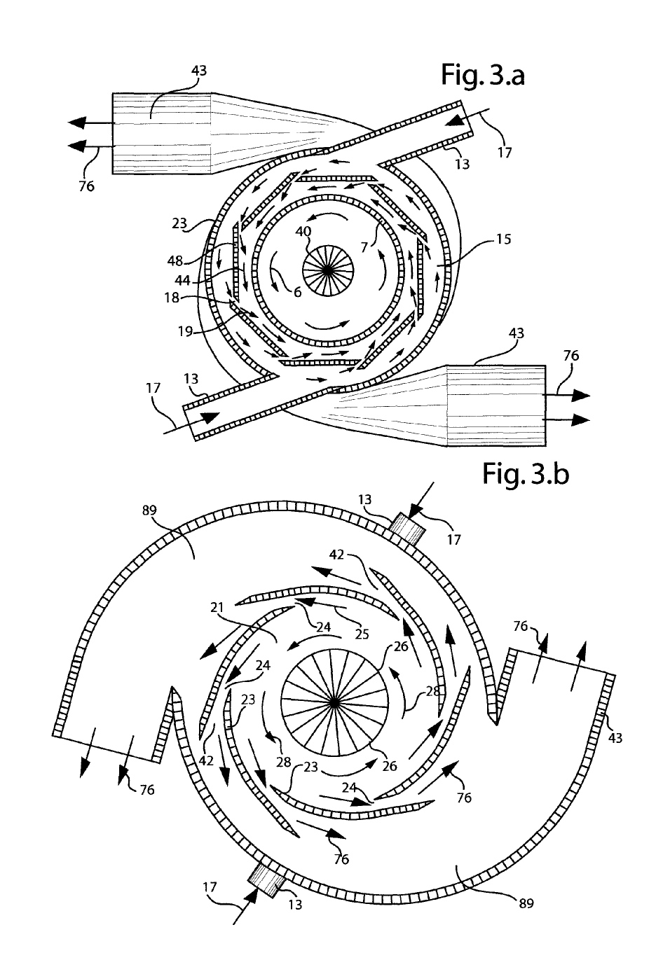

The static pressure drop of the fluids discharged (25) at high velocity may be partially recovered downstream of the tangential outlet openings (24), with the aid of conduits with flared outlets (42), which enable some of their dynamic pressure to be recovered, as shown in FIG. 3.b.

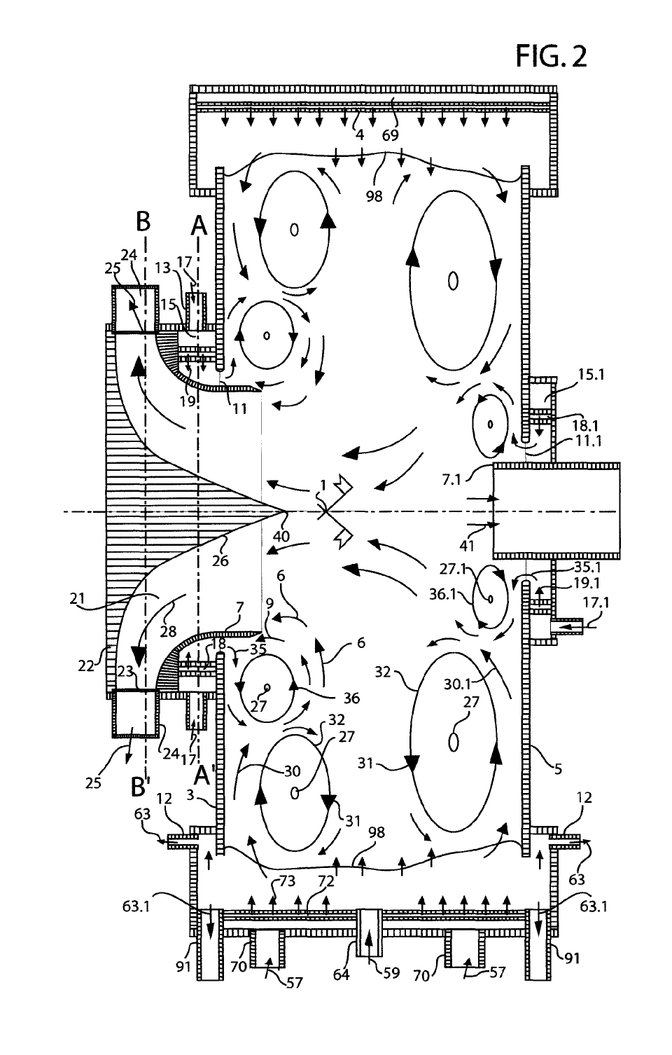

FIG. 2 shows an axial section through another example of devices for injecting secondary fluids, in which said injection openings (18) and (18.1) are located on the outside of the cyclone chamber, along the side walls (3) and (5), and with a central fluid discharge tube (7) and (7.1) on each lateral side of said cyclone chamber. In this example, the solid particles (59) are supplied through the tube (64) that passes through the middle of the peripheral wall (4). They are entrained in a rotary movement by the fluid (57) injected in a mainly tangential direction (73) into the cyclone chamber. They are added to the solid particles retained in the middle of the cyclone chamber, and are pushed axially by centrifugal force toward the side walls (3) and (5).

The heavy solid particles are discharged via two decantation tubes (91) located near the side walls (3) and (5). Rotary valves or pairs of valves opening alternately, not shown in the figure, enable the pressure differences to be absorbed without entraining the fluids revolving in the cyclone chamber, and enable the flow rate of the heavy solid particles to be controlled.

The other solid particles, mainly the finest or lightest ones, are discharged via the outlet tubes (12) which determine the thickness of the rotating fluidized bed whose free surface is represented by the wavy line (98). Said other solid particles and the fluid flow that entrains them in the tubes (12) may be separated by a suitable device such as a cyclone or filter.

The injection openings (18) and (18.1) of the devices for supplying the secondary fluids (17) and (17.1) are located, in this example, outside the cyclone chamber. Said rotating rings of secondary fluids, formed behind said side walls, penetrate into the cyclone chamber through annular openings (11) and (11.1), which are preferably narrow, between said central tubes (7) and (7.1) and said side walls (3) and (5), which act as the guide walls (38) and (38.1) of FIG. 1. This separation enables said rotating rings of secondary fluids to be made uniform before coming into contact with the fluids revolving in the cyclone chamber, to avoid generating undesirable turbulence.

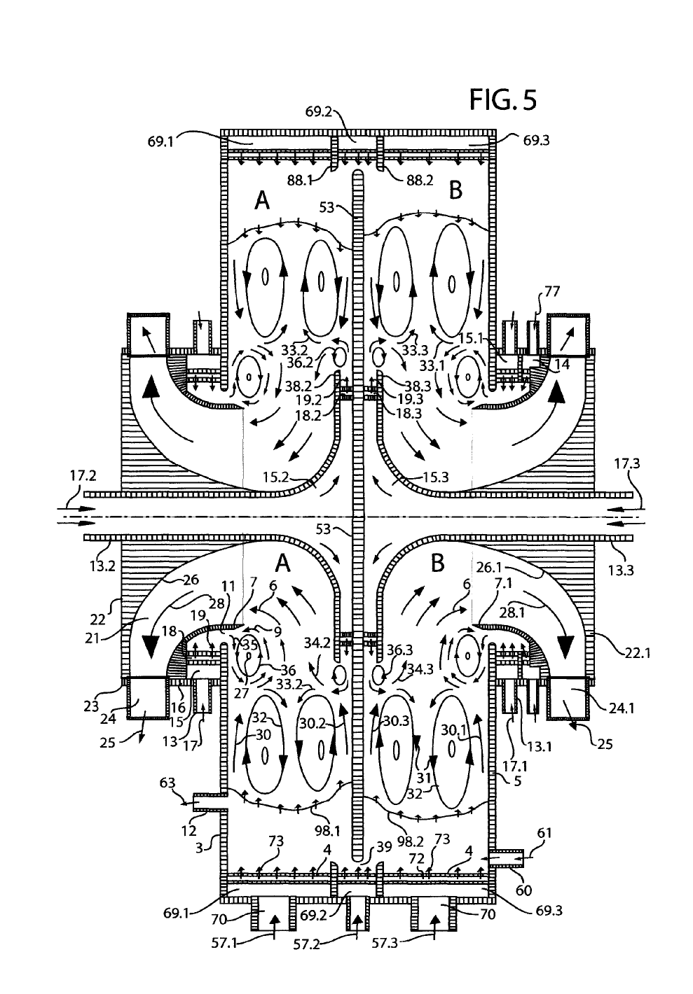

The second central tube (7.1) for discharging the fluids is relatively small in this example, so that it mainly discharges the central backflow (41) originating from the virtually non-rotating area that lies along the axis of cylindrical symmetry (1). This central tube (7.1) may be similar to the central tube (7), as in FIG. 5.

FIG. 3.a shows a transverse section, along the cutting line AA′ of FIG. 2, through the device for injecting secondary fluids (17) which are supplied under pressure via the supply tubes (13) into the central supply chamber (15) and injected through eight injection openings (18), delimited by eight successive plates (48). These secondary fluids, injected in a mainly tangential direction (19) at high velocity around the central tube (7), form a rotating ring (44) of secondary fluid between said successive plates (48) and said central tube (7) outside the side wall (3), before penetrating into the cyclone chamber. Said rotating ring is represented by the numbers (44.1), (44.2) and (44.3) respectively when it relates to the secondary fluids (17.1), (17.2) and (17.3).

The centrally exiting fluid flows (6) revolve inside the central tube (7) around the section of the tip (40) of said curved surface of said lateral disk. They are discharged by two outlet tubes (43), which are shown for guidance in the background.

FIG. 3.b shows a transverse section, along the cutting line BB′ of FIG. 2, through the device for central discharge of the fluids, according to a preferred embodiment of the invention. The deflected fluids (28) revolve around the curved surface (26) inside the cylindrically symmetric conduit (21), and are discharged at high velocity through six tangential outlet openings (24) with small cross sections, connected to two manifolds (89) by flared conduits (42), whose outlet cross sections are preferably at least three times larger than the cross sections of the tangential outlets (24), in order to recover some of the dynamic pressure of the discharged fluids (25). The decelerated fluids (76) are discharged via the two tubes (43).

If the cross sections of the tangential outlet openings (24) are small enough, this device makes it possible to maintain, and even increase, the rotation velocity of the deflected fluids (28) and therefore of the centrally exiting fluids (6) in the central tube (7), so as to have a positive effect on the rotation velocity of the fluids in said cyclone chamber.

The tubes (13) for supplying secondary fluids (17) are shown, for guidance, in the background.

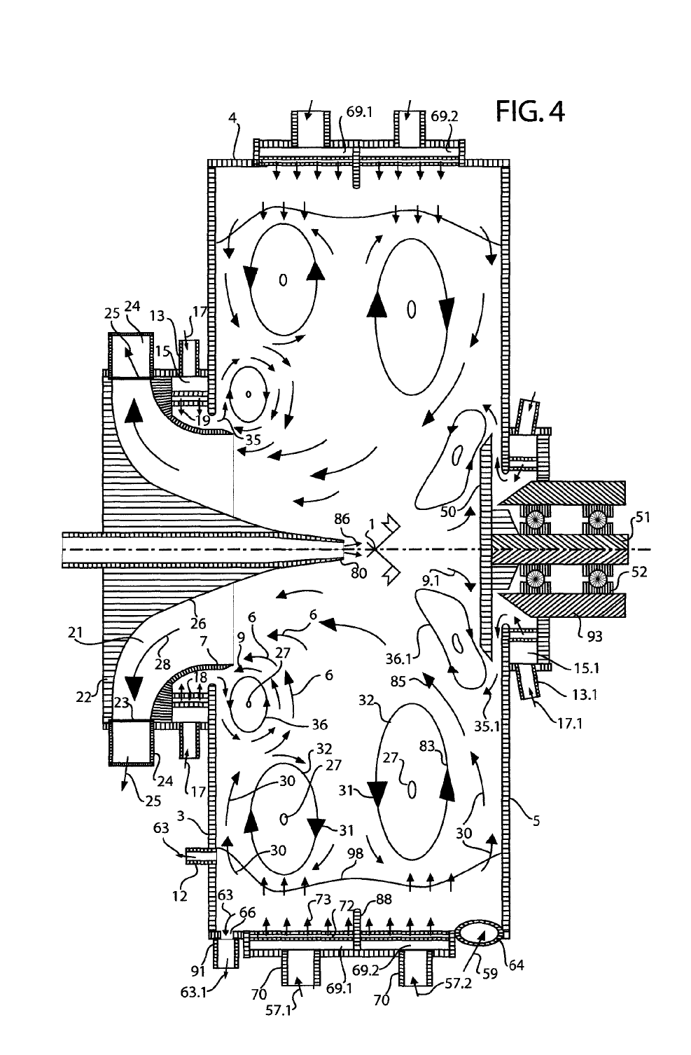

FIG. 4 shows another example of a cyclone chamber equipped with the devices for injecting secondary fluids, according to the invention, with a rotating lateral disk (50), near the side wall (5), guiding the backflow of secondary fluid (35.1) and supported by a device symbolized by a rotating shaft (51) and ball bearings (52) held by a fixes support (93), enabling said rotating disk to be revolved at a sufficient velocity to accelerate the rotation velocity of the fluids revolving in the cyclone chamber near the rotating disk. The greater centrifugal force generated by the rotating disk also increases the intensity of the toroidal vortex (36.1), which retains the solid particles entrained by the exiting fluid flows (30.1) along the side wall (5). The diameter and rotation velocity of said rotating disk are chosen according to the requirements of the method using this device.

In this example, a tube (64) supplies solid particles (59) through said peripheral wall, these particles preferably being tangentially entrained by a fluid, near the side wall (5). The fluids (57.1) and (57.2), supplied via the tubes (70) into the supply chambers (69.1) and (69.2) surrounding the cyclone chamber, are injected tangentially via injection openings, such as longitudinal injection slots (72), to make said fluids and said solid particles (63) revolve in the cyclone chamber.

The coarser solid particles (63.1) are discharged through an outlet opening (66) along the circular wall (4) via a decantation tube (91), preferably located near the other side wall (3), and the lighter particles (63) are preferably discharged via a tube (12) through the side wall (3).

In this particular example, the central tube (7), passing through the side wall (3) opposite said lateral rotating disk (50), surrounds a concentric tube (80) which penetrates into the cyclone chamber near the lateral rotating disk (50). It may be used for centrally supplying a fluid (86), which may, for example, be atomized in the form of droplets of a liquid used, for example, for cooling the fluids or for impregnating the solid particles revolving in the cyclone chamber.

This example also comprises an annular separating wall (88) which may be used to reduce the axial flow of the solid particles revolving along the circular wall (4) between said annular separating wall and the side walls (3) and (5), according to the requirements of the method using this device.

FIG. 5 shows an axial section through another example of an approximately symmetrical cyclone chamber, divided into two transverse sections A and B by a separating wall (53), and having devices for injecting secondary fluids along the two sides of said separating wall.

The fluids, (57.1) to (57.3), are supplied through three supply chambers, (69.1) to (69.3), surrounding the cyclone chamber. The fluid (57.2) is injected through openings (72) distributed to face the periphery of said separating wall (53), which is located at a short distance from said peripheral wall, leaving a passage (39), which is preferably narrow, allowing the transfer of the solid particles from one said transverse section to the other. Annular separating walls (88.1) and (88.2) may guide the fluid (57.2) on either side of said separating wall, in order to strip the flow of solid particles, transferred from one transverse section to the other, from the fluids that entrain them.

In this example, the passage (39) covers the whole periphery of said separating wall and the openings (72) for supplying the fluid (69.2) are aligned along said passage (39). The flow of the solid particles between the two transverse sections is generated by the turbulence. This flow may be increased by using multiple passages (39), arranged alternately facing the upstream end of an opening (72) on one side, and facing the downstream end of an opening (72) on the other side, of said separating wall.

The solid particles (61) are supplied via a supply tube (60) passing through the side wall (5), and the solid particles (63) are discharged via a tube (12) passing through the side wall (3) at a distance from the peripheral wall (4) which determines the mean level of the free surface (98.1) of the rotating fluidized bed revolving in said transverse section A. The mean of the free surface (98.2) of the rotating fluidized bed revolving in said transverse section B is determined by the pressure difference between the free areas of the two transverse sections. A suitable device for controlling the pressure differences, not shown, should enable the free surface (98.2) to be kept at the desired mean level.

The secondary fluids (17.2) and (17.3) are supplied via the tubes (13.2) and (13.3) which are concentric with the central tubes (7) and (7.1), and are injected via the openings (18.2) and (18.3) in mainly tangential directions (19.2) and (19.3) behind the guide walls (38.2) and (38.3), in order to form rotating rings of secondary fluids that accelerate the rotation velocity and push back the exiting fluid flows (30.2) and (30.3), which entrain solid particles along the surfaces of said separating wall (53), in order to retain toward said peripheral wall said retained parts (33.2) and (33.3) which contain most of the solid particles entrained by said exiting fluid flows (30.2) et (30.3). Said discharged parts (34.2) and (34.3) have practically ceased to contain any solid particles.

In this example, the device for supplying secondary fluids around the central tube (7.1) passing through the side wall (5) comprises a chamber (14) contiguous to the central supply chamber (15.1), allowing the supply of a different secondary fluid (77), one of which may be gaseous and the other liquid, in order to atomize said liquid inside the cyclone chamber. Said liquid may react chemically and/or physically with the exiting fluids (30.1), which are usually gases, and/or the solid particles that are entrained by said exiting fluids before being retained. The other three devices for supplying secondary fluids may also have such a device in the form of a contiguous central supply chamber.

The various devices and combinations of devices in the examples described above may be used by methods for processing solid particles reacting in contact with the fluids revolving in a cyclone chamber, such as combustion, gasification, pyrolysis, roasting, drying, encapsulation, surface treatment, impregnation, extraction of volatiles, oxidation and reduction, etc., of solid particles, and for methods for processing fluids revolving in a cyclone chamber in contact with the solid particles which may be catalysts and/or reactants, for example cracking, disproportionation, dehydrogenation, oxidation, oxidation and reduction, polymerization, etc., of fluids revolving in the cyclone chamber, said processing methods comprising the separation of said solid particles and said fluids.

The use of the device for injecting secondary air and for centrally discharging fluids according to the invention by said methods for processing solid particles and fluids revolving in a cyclone chamber is characterized in that the injection of secondary fluids makes it possible to improve the separation of the solid particles and the exiting fluids, by increasing the rotation velocity of the fluids in the free area of the cyclone chamber, mainly along the side walls and separating walls, without significantly accelerating rotation velocity of said rotating fluidized bed.

This device is therefore particularly useful for methods in which the desired ratio between the hourly mass flow rate of the fluids and the mass of the rotating fluidized bed is too small to allow a sufficient transfer of kinetic moment of rotation to make the rotating fluidized bed revolve at the necessary velocity to provide good separation between the fluids and the solids, or when the mechanical or physical constraints, for example the abrasion or the sensitivity of the solid particles to friction limit the rotation velocity of said solid particles along said peripheral wall, as in the example of the polymerization of polyethylene powders which is sensitive to the formation of angel hair.

The invention also relates to methods using the devices for injecting secondary fluids according to the invention, characterized in that at least one of said secondary fluids cools, or reacts chemically with, at least one of said exiting fluid flows that have reacted with the solid particles of the fluidized bed revolving in the cyclone chamber.

For example, gases resulting from gasification, or from the pyrolysis or roasting or extraction of volatiles from solid particles in suspension in the rotating fluidized bed, may be cooled quickly by the atomization of secondary liquids supplied by contiguous chambers as described in FIG. 5, to avoid undesirable secondary reactions.

FIG. 2 illustrates an example of a particular two-step method of combustion of carbonaceous, solid or fluid materials, such as coal dust or shredded organic waste, using oxygen-enriched gas, for example, characterized in that partial combustion is initially carried out in the rotating fluidized bed of said cyclone chamber, after which the oxygen of said secondary fluid injected by the injection device according to the invention terminates the combustion of the exiting gas flows (30) and (30.1).

The use of oxygen-enriched gas makes it possible to reduce the cost of recovering undesirable products such as CO₂. This combustion may be very rapid, while generating very high temperatures contributing to slag formation. Cyclone chambers with rotating fluidized beds enable virtually explosive combustion to be controlled because of the very high ratio of the mass flow rates of the gases to the mass of the solids presents in the rotating fluidized bed, which may be greater than 1000 per hour, owing to a centrifugal force which is higher by more than an order of magnitude than the force of gravity, and also owing to the large surface and thinness of the rotating fluidized bed.

For guidance, a 1 m³ cyclone chamber may contain a rotating fluidized bed containing about 100 kg of powder, and may have an hourly mass flow rate of pressurized gas of more than 200 tonnes per hour passing through it, enabling carbon to be partially burnt at about 10 kg per second, with a mixture by mass of about ⅔ vapor and ⅓ pure oxygen. If the rotating fluidized bed contains 20% coal dust, the remainder being ashes or inert, oxidizing or catalytic powders, the partial combustion time for the coal dust is about two seconds. The combustion of gases rich in CO may be completed at the time of their exit from the cyclone chamber by the oxygen contained in the secondary fluids, which are injected at high velocity into the free area of the cyclone chamber.

Carbonaceous matter (59) may, for example, be supplied in suspension, or dissolved in a liquid, for example water, using a high-pressure pump. The mixture may be preheated to a temperature allowing some of the liquid to vaporize, to generate a mixture of solids, liquid and vapor (59) which may be injected tangentially, at high velocity, into the cyclone chamber via the tube (64). A gas containing an insufficient quantity of oxygen to provide complete combustion of all the carbon contained in said carbonaceous matter is supplied by the supply chambers (69.1) and (69.2) into the cyclone chamber which is preheated to a sufficient temperature to burn the carbon, producing a mixture of CO and CO₂. The flow rates may be adjusted to obtain the desired temperature, which is sufficiently high for rapid combustion and sufficiently low to avoid slag formation, being about 650° C. for example, in the peripheral part of the cyclone chamber.

The heavy ashes (63) may be discharged through said peripheral wall via the decantation tubes (91). The fine or light ashes (87) are discharged via the lateral outlets (12), or are entrained along the side walls (3) and (5) by the exiting fluid flows (30) and (30.1) and retained by the backflows of secondary fluids (35) and (35.1), which form the secondary toroidal vortices (36) and (36.1), toward said peripheral wall.

The oxygen contained in the secondary fluids (17) and (17.1) may terminate the oxidation of the partially oxidized gases in the central part of the cyclone chamber and in the central tubes, without allowing the heat evolved by this post-combustion to overheat the ashes formed along the circular wall. The light ashes that have been retained by the secondary vortex may be very hot and may form, in the peripheral part of the cyclone chamber, small clusters that may be discharged via the decantation tubes (91).

Annular separating walls (88), as illustrated in FIGS. 4 and 5, may be added to separate the rotating fluidized bed into a plurality of sections through which fluids having different compositions and/or temperatures pass. For example, water vapor may be injected around the decantation tubes to strip the heavy particles before discharging them.

The small size of such a cyclone chamber allows operation at very high pressure, and the post-combustion makes it possible to reach the temperatures required for the complete combustion of the undesirable components and the efficient recovery of the combustion energy. The use of pure oxygen and carbonaceous matter in suspension, or dissolved in water, makes it possible to control the temperature of the rotating fluidized bed and to recover the CO₂ at lower cost. Finally, the quality of the separation of the burnt gases and solid particles, due to the central injection of very fast secondary gases, generating a centrifugal force that may be greater by several orders of magnitude than gravity in the central part of the cyclone chamber, may retain the fine solid particles to a sufficient extent to allow a turbine to be supplied directly.

The device described by FIG. 4, with or without a rotating disk, may be used, for example, for the methods of encapsulation, surface treatment or impregnation. The encapsulation, surface treatment or impregnation liquid (86) may be atomized by the tube (80) onto the rotating disk (50) that revolves at high velocity, or may be sent by means of an atomizer directly into the free area of the cyclone chamber. The liquid may also be injected via atomizers passing through a side wall or the peripheral wall, or by means of a contiguous central supply chamber (14), illustrated in FIG. 5.

The device according to the invention also relates to methods for processing solid particles such as surface treatment, encapsulation and impregnation, and is characterized in that it comprises at least one contiguous central supply chamber for supplying a secondary liquid that reacts with the solid particles entrained by a said fluid flow exiting along a said side wall or separating wall.

The centrifugal force pushes the droplets of liquid toward the peripheral wall where they may react with the solid particles in suspension in said rotating fluidized bed. The solid particles then migrate toward the side wall (3), on the other side of the cyclone chamber, along the circular wall (4) through which the gases (57.1) and (57.2) pass. The composition and temperature of these gases are chosen according to the requirements of the method. For example, the gas (57.2) may be cold to retard drying, in order to make a surface treatment uniform, or may be hot in order to accelerate the setting of the surface treatment as the treatment progresses. The solid particles are then discharged via the tube (12) or the decantation tube (91). An annular wall (88) may increase the dwell time of the solid particles in the transverse section on the right of the cyclone chamber.

The device according to the invention also relates to methods for processing fluids and solid particles comprising the alternating processing of said solid particles in a cyclone chamber divided into two transverse sections according to one of the particular embodiments of the invention, and is characterized in that it comprises an endothermic reaction in one section and an exothermic reaction in the other section, and in that at least one of said secondary fluids reacts with at least one of said exiting fluid flows.

The device described by FIG. 5 illustrates the methods of alternating processing, for example an oxidation step that regenerates catalytic solid particles and/or generates the heat required for the second step, for example catalytic cracking or gasification or dehydrogenation, which absorbs heat. The solid particles are supplied through the side wall (5) via the tube (68) and are discharged on the other lateral side via the tube (12). They form a rotating fluidized bed through which the fluid (57.3), hydrocarbons for example, passes from one side, while the fluid (57.1), air for example, passes through it from the other side. A separating fluid (57.2), water vapor for example, may be used to strip the solid particles of the fluids that they entrain with them when they pass from one side to the other.

When the device is in operation, the solid particles form a rotating fluidized bed and flow rapidly from one side of the bed to the other, while transferring their heat and their kinetic moment of rotation, thereby contributing to a substantial reduction in the differences between the temperatures and rotation velocities on the two sides of the rotating fluidized bed. On the other hand, the fluids pass through the rotating fluidized bed very rapidly and are discharged separately via the central discharge device located on their side, without mixing with one another, the separating wall providing suitable separation. The separation fluid (57.2) may be used to improve this separation by preventing the transfer of the other fluids from one side to the other. It may also be injected at very high velocity to increase the rotation velocity of the rotating fluidized bed.

For example, for a gasification method, the fluids (57.3) and (57.2) may be water vapor and the fluid (57.1) may be air. The carbonaceous particles are introduced through the side wall (5) with other solid particles, such as dolomite, that serve to improve the heat transfers and may also serve to catalyze or prevent the formation of slag.

The rotating fluidized bed is heated by the combustion of the residual carbonaceous matter in contact with the air in section A of the cyclone chamber. This heat is transferred by the rotating fluidized bed to section B where the carbonaceous particles are introduced, in order to reach the necessary temperature for gasification.

Another example of a method that may use a device similar to that described in FIG. 5 is the dehydrogenation of hydrocarbons such as ethane or propane to produce olefins, and the dehydrogenation of ethylbenzene to produce styrene by processes of oxidation and reduction, or oxidation, of carbonaceous matter using catalysts containing metallic oxides which are reduced in one section of the cyclone chamber and reoxidized in the other section, through which an oxidizing fluid passes. The heat produced by the oxidation in said other section is transferred by the rotating fluidized bed into the part where the oxygen transfer, which is an endothermic reaction, takes place.

1. A device for injecting secondary fluids into the free area of a rotating fluidized bed in a fixed cyclone chamber, comprising a peripheral wall (4) surrounded by two side walls (3) and (5); a device for supplying fluid (57) through openings (72) distributed along said peripheral wall, in a mainly tangential direction (73); a device for supplying and discharging solid particles; and at least one central tube (7) for discharging the fluids revolving in said cyclone chamber, characterized in that it comprises:
a device for supplying secondary fluids (17) and (17.1) through central supply chambers (15) and (15.1) located along each of said side walls (3) and (5), having injection openings (18) and (18.1) distributed around the axis of cylindrical symmetry, enabling said secondary fluids to be injected in a mainly tangential direction (19) and (19.1), in order to form two rotating rings of secondary fluids revolving around said axis of cylindrical symmetry, along said side walls (3) and (5), within said free area, and in that said devices for supplying said secondary fluids enable said secondary fluids to be supplied at a sufficient pressure to make said rotating rings of secondary fluids revolve at a velocity which is greater than the highest rotation velocity of said fluids revolving in cyclone chamber when the latter is in operation without the device for injecting secondary fluid according to the invention.

2. The device as claimed in claim 1, characterized in that said cyclone chamber has a width smaller than its diameter, and in that the distance between the openings (72) for supplying fluid (59) through said peripheral wall is smaller than its mean radius.

3. The device as claimed in claim 1, characterized in that it comprises at least one said central tube for discharging the fluids from each lateral side of the cyclone chamber.

4. The device as claimed in claim 3, characterized in that it comprises at least one separating wall (53), dividing the cyclone chamber into two transverse sections A and B, said separating wall having at least one passage (39) allowing the passage of solid particles entrained by the fluids along said peripheral wall (4) from one said transverse section to the other, and in that it comprises a device for injecting secondary fluids (17.2) and (17.3) along the two sides of said separating wall, in a mainly tangential direction, into said free areas of said transverse sections.

5. The device as claimed in claim 4, characterized in that it comprises said passages (39) located alternately facing the upstream end of an opening (72) on one side, and facing the downstream end of an opening (72) on the other side, of said separating wall.

6. The device as claimed in claim 1, characterized in that at least one said rotating ring of said secondary fluids is formed behind a guide wall (38) before coming into contact with the fluid flows revolving in said free area of the cyclone chamber.

7. The device as claimed in claim 1, characterized in that at least one device for injecting secondary fluid may supply at least 8% of the flow rate of fluids passing through the cyclone chamber, and comprises at least eight said injection openings distributed in said central supply chamber.

8. The device as claimed in claim 1, characterized in that at least one said central tube (7) terminates in a cylindrically symmetric conduit (21), delimited on one side by the flared surface (20) of the end of said central tube (7), and on the other side by the curved surface (26) of a lateral disk (22) closing said central tube (7); said cylindrically symmetric conduit being closed by a circular wall (23) whose inside width (49) is less than the radius of the inlet of said central tube (7), said circular wall (23) having at least two tangential outlet openings (24) for the tangential discharge of the fluids (28) deflected by said cylindrically symmetric conduit.

9. The device as claimed in claim 8, characterized in that the number of said tangential outlet openings (24) is at least four, and in that the sum of their cross sections is at least twice as small as the cross section of said central tube (7).

10. The device as claimed in claim 1, characterized in that a said device for supplying secondary fluids (17) comprises two said contiguous central supply chambers (15.1) and (14), of which one may be used to supply a secondary gas and the other may be used to supply a secondary liquid.

11. The device as claimed in claim 1, characterized in that it comprises at least one tube (80) concentric with a said central tube (7), penetrating into the cyclone chamber and enabling droplets of a liquid to be atomized in said cyclone chamber.

12. The device as claimed in claim 1, characterized in that the device for discharging the solid particles comprises at least one outlet opening (66) in the peripheral wall (4) of the cyclone chamber, surrounded by a decantation tube (12) for the accumulation and discharge of the solid particles (63) under the effect of their inertia and the force of gravity when the device is operating.

13. Use of the device as claimed in claim 1 by a method for processing fluids and solid particles revolving in a cyclone chamber, comprising the separation of said solid particles and said fluids, characterized in that said fluid flows exiting while entraining solid particles along said side walls are separated from said side walls, and their rotation velocity is accelerated by the injection of said secondary fluids along said side walls.

14. A method for processing solid particles and fluids revolving in a cyclone chamber comprising a device for injecting secondary fluid as claimed in claim 1, characterized in that at least one of said secondary fluids reacts with at least one of said fluid flows exiting along a said side wall or separating wall.

15. The method for processing solid particles and fluids revolving in a cyclone chamber as claimed in claim 14, characterized in that it comprises at least two said contiguous central supply chambers, one injecting a secondary liquid and the other injecting a secondary gas, said secondary liquid reacting with the solid particles entrained by a said fluid flow exiting along a said side wall or separating wall.

16. A method for the combustion of carbonaceous matter revolving in a cyclone chamber as claimed in claim 14, characterized in that the fluids injected tangentially through the circular wall (4) do not contain enough oxygen to provide complete combustion of said carbonaceous matter, and in that at least one said secondary fluid supplied by a device according to the invention contains the necessary oxygen to complete the oxidation of said fluid flows exiting along a side wall.

17. A method for processing fluids and solid particles revolving in a cyclone chamber comprising a device for injecting secondary fluid and divided in two transverse section A and B by a separating wall as claimed in claim 4, characterized in that it comprises an endothermic reaction in one section and an exothermic reaction in the other section, and in that at least one of said secondary fluids reacts with at least one of said exiting fluid flows.
